(19) United States
(12) Patent Application Publication
(10) Pub. No.: US 2010/0280077 A1
(43) Pub. Date: Nov. 4, 2010

(54) Process for Preparation of Stable Amorphous R-Lansoprazole

(75) Inventors: Samir Naik, Thane (IN); Arjun Bodke, Shantinagar (IN); Kishor Mahajan, Dombivli (West) (IN); Manjunath Narayan Bhanu, Pune (IN)

Correspondence Address: Florek & Endres PLLC, 1156 Avenue of the Americas, Suite 600, New York, NY, 10036, US

(73) Assignee: WATSON PHARMA PRIVATE LIMITED, Andheri (East), Mumbai (IN)

(21) Appl. No.: 12/808,351

(22) PCT Filed: Dec. 18, 2008
(86) PCT No.: PCT/IN2008/000842

§ 371 Date: Jun. 15, 2010

(30) Foreign Application Priority Data
Dec. 18, 2007 (IN) 2489/MUM/2007

(51) Int. Cl. A61K 31/4439; C07D 401/12; A61P 1/04

(52) U.S. Cl. 514/338; 546/273.7

(57) ABSTRACT

A process for preparing stable amorphous R-(+)-lansoprazole comprising optically resolving racemic lansoprazole by the formation of host-guest inclusion complexes by selectively and reversibly including chiral guest molecules in the lansoprazole.

### FIELD OF THE INVENTION

The present invention relates to a process for preparation of stable amorphous (R)-2-[[[3-methyl-4-(2,2,2-trifluoroethoxy)-2-pyridinyl]methyl]sulfinyl]-1H-benzimidazole, also known as R-(+)-lansoprazole.

### BACKGROUND OF THE INVENTION

2-[[[3-methyl-4-(2,2,2-trifluoroethoxy)-2-pyridinyl]methyl]sulfinyl]-1H-benzimidazole, also known as lansoprazole, is disclosed in Japanese patent application No. JP-A-61-50978.

Lansoprazole is a well-known gastric acid secretion inhibitor and is useful as an anti-ulcer agent. Lansoprazole has a chiral sulfur within its molecular structure and hence occurs as two optical isomers, R-lansoprazole and S-lansoprazole.

U.S. Pat. No. 6,462,058 B1 discloses a crystal of R-lansoprazole and its use as an anti-ulcer agent. U.S. Pat. Nos. 6,462,058 B1 and 6,664,276 B2 and PCT Patent Publication No. WO 00/78745 A2 all describe the synthesis of a crystal of R-lansoprazole. Exemplary methods for such synthesis include:

a) Optical resolution of lansoprazole by a fractional crystallization method, which includes forming a salt between a racemate and an optically active compound [for example, (+)-mandelic acid, (−)-mandelic acid, (+)-tartaric acid, or (−)-tartaric acid]. The diastereoisomeric salt is separated by fractional crystallization and then subjected to a neutralization process to give a free optical isomer.

b) The chiral column method includes a method in which a racemate or a salt is applied to a column for optical isomer separation. In liquid chromatography, for example, optical isomers are separated by adding the racemate to a chiral column (such as the Daicel series (produced by Daicel Chemical Industries, Ltd.), and eluting in water, a buffer (for example, a phosphate), an organic solvent (for example, hexane, ethanol, methanol, isopropanol, acetonitrile, triethylamine, or mixtures thereof) or mixtures of the foregoing.

c) The asymmetric oxidation process includes subjecting lansoprazole to an asymmetric oxidation to obtain (R)-(+)-lansoprazole, followed by crystallizing the resultant isomer.

The above-mentioned patents and patent applications also describe the X-ray powder diffraction characteristics of the crystal of R-(+)-lansoprazole and the use of crystalline R-(+)-lansoprazole for manufacturing a pharmaceutical composition for the treatment or prevention of a digestive ulcer.

Generally, amorphous compounds exhibit better solubility and much higher bioavailability than their crystalline counterparts. It is an object of the present invention to provide a process for the synthesis of stable amorphous R-(+)-lansoprazole.

### SUMMARY OF THE INVENTION

According to one aspect, the present invention relates to a stable amorphous form of R-(+)-lansoprazole.

According to another aspect, the present invention relates to a process for preparation of a stable amorphous R-(+)-lansoprazole, which includes:

i) Optical resolution of racemic lansoprazole by the formation of host-guest inclusion complexes via selectively and reversibly including chiral guest molecules in the host lattices of chiral molecules;

ii) Resolving lansoprazole with 2,2′-dihydroxy-1,1′-binaphthyl (BINOL) as the chiral host, by forming the inclusion complex in a suitable solvent system such as a mixture of toluene and hexane;

iii) Crystallizing the inclusion complex from a suitable solvent system such as a mixture of toluene and hexane to enrich the R-isomer to more than 97% enantiomeric excess (“e.e.”), otherwise known as optical purity;

iv) Cleaving the R-(+)-lansoprazole-BINOL inclusion complex with a suitable solvent such as an aqueous ammonia solution and isolating R-(+)-lansoprazole as an amorphous material;

v) Forming a complex of R-(+)-lansoprazole with a pharmaceutically acceptable polymer such as polyvinylpyrrolidone; and

vi) Isolating R-(+)-lansoprazole as a complex with the pharmaceutically acceptable polymer in a stable amorphous form.

### DESCRIPTION OF THE INVENTION

One aspect of the present invention is to provide a process for resolution of racemic lansoprazole, to R-(+)-lansoprazole comprising:

a) Treating racemic lansoprazole with R-(+)-BINOL (R-(+)-2,2′-dihydroxy-1,1′-binaphthyl) in a suitable solvent to form the inclusion complex of R-(+)-lansoprazole with R-(+)-BINOL;

b) Removing the inclusion complex of R-(+)-lansoprazole with R-(+)-BINOL prepared in step (a) from the solvent and adding a mixture of organic solvents, such as toluene and hexane, to the inclusion complex at a temperature of about 10° C. to about 40° C., more preferably about 20° C. to 25° C.;

c) Crystallizing the R-(±)-lansoprazole-R-(+)-BINOL inclusion complex from the mixture of organic solvents at a temperature of about −5° C. to about 30° C., more preferably about 0° C. to about 10° C. and most preferably about 0° C. to about 5° C.;

d) Enriching the R-(+)-lansoprazole-R-(+)-BINOL inclusion complex, preferably by recrystallization from a suitable solvent system such as a mixture of toluene and hexane to give R-(+)-lansoprazole-R-(+)-BINOL inclusion complex having a chiral purity of more than about 97% e.e;

e) Cleaving the enriched R-(+)-lansoprazole-R-(+)BINOL complex by treating with a suitable solvent system such as liquor ammonia at about 0° C. to about 50° C., preferably about 20° C. to about 45° C. and more preferably about 25° C. to about 35° C., to obtain a mixture of R-BINOL and R-(+)-lansoprazole in aqueous ammonia solution;

f) Separating out R-BINOL from R-(+)-lansoprazole; and

g) Isolating R-(+)-lansoprazole from the aqueous ammonia solution by adjusting the pH of the ammonia solution from about 7 to about 10, more preferably about 8 to about 9.5 and most preferably about 9 to about 9.3, and filtering the isolated R-(+)-lansoprazole.

Another aspect of the present invention provides a process for preparing a stable amorphous R-(+)-lansoprazole comprising:

1) Dissolving R-(+)-lansoprazole in a suitable solvent such as ethanol and adding about 5% to about 40% of a pharmaceutically acceptable polymer such as polyvinylpyrrolidone;

2) Concentrating the above compound under a vacuum to dryness to obtain stable amorphous R-(+)-lansoprazole adsorbed on the pharmaceutically polymer.

The racemic lansoprazole employed as the starting material for the present invention can be obtained by any conventional process known in the art such as the process described in the aforementioned Japanese Patent Application No. JP-A-61-50978.

The solvent used to form the inclusion complex of R-(+)-lansoprazole with R-(+)-BINOL in step (a) is preferably an organic solvent preferably a halogenated organic solvent such as methylene dichloride. Once the inclusion complex of R-(+)-lansoprazole with R-(+)-BINO is prepared the solvent used in the preparation step (a) is removed by conventional techniques such as by distillation or evaporation. If the preparation solvent of step (a) is methylene dichloride, the preferred removal method is by distillation.

Once the preparation solvent of step (a) is removed, a mixture of organic solvents is added to the inclusion complex. The mixture of organic solvents preferably is a mixture of aromatic and aliphatic solvents such as toluene and hexane. The ratio of aromatic to aliphatic in the mixture should range from about 1:1 to about 5:0.5, preferably about 2:1 to about 5:1 and most preferably about 4:1.

The enrichment of the R-(+)-lansoprazole-R-(+)-BINOL inclusion complex can be performed by any conventional methods, preferably by one or more recrystallizations from a suitable solvent system. A suitable solvent system is preferably mixture of organic solvents. The preferred mixture comprises a combination of aromatic and aliphatic solvents such as toluene and hexane. The ratio of aromatic to aliphatic in the mixture should range from about 1:1 to about 5:0.5, preferably about 2:1 to about 5:1 and most preferably about 4:1. Once the enrichment step is completed the R-(+)-lansoprazole-R-(+)-BINOL inclusion complex should have purity of at least 97% and preferably at least 98%.

After the R-(+)-lansoprazole-R-(+)-BINOL inclusion complex is enriched, the complex is cleaved using a suitable solvent cleaving system. A suitable solvent cleaving system may contain water and an amine compound. A preferred solvent cleaving system is an aqueous ammonia solution. Once the R-(+)-lansoprazole and R-(+)-BINOL have been cleaved, the R-(+)-lansoprazole and BINOL components are separated by methods known in the art. In one embodiment of the present invention, the R-(+)-lansoprazole and the BINOL components are separated by solvent extraction. The preferred solvent for the extraction is methyl tertiary butyl ether.

Following the separation, the amorphous R-(+)-lansoprazole is collected and further stabilized by dissolving the R-(+)-lansoprazole in a suitable solvent, preferably an alcohol such as ethanol and adding about 5% to about 40% of a pharmaceutically acceptable polymer based upon the weight of the R-(+)-lansoprazole. The pharmaceutically acceptable polymer is preferably a water soluble polymer. The pharmaceutically acceptable polymer should exhibit a viscosity of less than 200 mPa s, preferably less than 100 mPa s and most preferably less than 50 mPa s when a 5% m/v aqueous preparation is prepared. One embodiment of the present invention employs polyvinylpyrrolidone as the pharmaceutically acceptable polymer.

The stable amorphous R-(+)-lansoprazole prepared in accordance with the present invention may be mixed with at least one, additional conventional pharmaceutical excipient to prepare a pharmaceutical dosage form such as a tablets, capsule or solution.

The following are provided as examples of the invention are not intended to be limiting.

### EXAMPLES

### Example 1

### Resolution of (R)-2-[[[3-methyl-4-(2,2,2-trifiuoroethoxy)-2-pridinyl-1]methyl]sulfinyl]-1H-benzimidazole (R-(+)-Lansoprazole) from racemic Lansoprazole.

2-[[[3-methyl-4-(2,2,2-trifiuoroethoxy)-2-pridinyl-1]methyl]sulfinyl]-1H-benzimidazole (racemic Lansoprazole) (200 gm, 0.542 moles) and R-(+)-BINOL (232.52 gm, 0.813 moles) was dissolved in methylene dichloride (7 L) at room temperature in a 10 L flask and heated to 35-40° C., under stirring, to get a clear solution. The reaction mixture was concentrated under vacuum below 35° C. until the volume of the reaction mixture was approximately 5-6 times with respect to lansoprazole. Toluene (5760 ml) was charged to the reaction mixture, which was cooled to about 20-25° C. n-Hexane (1440 ml) was then added and the temperature was maintained between 20° C. and 25° C. The reaction mixture was then cooled to 0-5° C. and stirred at this temperature for 12 hours. The crystallized product was filtered and washed with n-hexane and suck-dried to obtain 230 gm of crude R-(+)-lansoprazole-R-(+)-BINOL inclusion complex.

Purification of Crude Complex

The crude R-(+)-lansoprazole-R-(+)-BINOL inclusion complex was dissolved in 3000 ml methylene dichloride and concentrated under vacuum maintaining the temperature below 35° C. until the volume of the reaction mixture was about 5-6 times with respect to lansoprazole. Toluene (5760 ml) was charged to reaction mixture and the reaction mixture was cooled to about 20-25° C. n-Hexane (1440 ml) was added maintaining the temperature between 20° C. and 25° C. The reaction mixture was then cooled to 0-5° C. and stirred at this temperature for 12 hours. The crystallized product was filtered and washed with n-hexane. The wet product was dried at 40° C. under vacuum to get 185 gm of pure R-(+)-lansoprazole-R-(+)-BINOL inclusion complex

Chiral purity by HPLC>97% ee

Yield: 92.55% w/w

### Example 2

### Preparation of (R)-2-[[[3-methyl-4-(2,2,2-trifluoroethoxy)-2-pridinyl-1]methyl]sulfinyl]-1H-benzimidazole (R-(+)-Lansoprazole)

185 gm of the pure R-(+)-lansoprazole-R-(+)-BINOL inclusion complex obtained in Example 1 was dissolved in methanol (92.5 ml) and liquor ammonia (925 ml) at 25-35° C. Methyl tertbutyl ether (MTBE) (925 ml) was added to the reaction mixture at 25-35° C. and stirred for 10 min to get a clear solution. The organic layer was separated and extracted with liquor ammonia twice (462.5 ml×2 times) at 25-35° C. and the organic layer was again separated. All of the aqueous layers were collected and washed twice with MTBE (462.5 ml×2) at 25-35° C. The aqueous layer was separated and cooled to 10° C. The pH of the aqueous layer was adjusted to 9-9.3 using 50% aqueous acetic acid solution. The reaction mass was cooled to 5° C. and stirred for 60 minutes maintaining the temperature between 5° C. and 10° C. The product obtained was filtered and washed with a mixture of 1 ml liquor ammonia solution in 200 ml chilled water and then with 100 ml chilled water. The wet product was dried at 40° C. under vacuum to get 59 gm of R-(+)-Lansoprazole

Chiral Purity by HPLC>97% e.e.

Yield: 32% w/w

### Example 3

### Preparation of Stable Amorphous R-Lansoprazole

6 gm of the R-(+)-lansoprazole obtained from example 2 was dissolved in 6 ml ethanol. A solution of 1.2 gm of polyvinylpyrrolidone (PVP, K-30) was prepared in 6 ml of ethanol and this solution was added to the R-(+)-lansoprazole solution slowly at 25-30° C. The reaction mixture was concentrated to dryness under vacuum maintaining the temperature below 35° C. The solid amorphous product obtained was dried at 25-35° C. under vacuum.

Yield: 120% w/w

We claim:

1. Amorphous R-(+)-lansoprazole.

2. A process for preparing stable amorphous R-(+)-lansoprazole comprising optically resolving racemic lansoprazole by forming a reversible host-guest inclusion complex that includes a chiral guest molecule in the lansoprazole lattice.

3. The process of claim 2 comprising the following steps:
(a) treating racemic lansoprazole with R-(+)-2,2′-dihydroxy-1,1′-binaphthyl (“R-(+)-BINOL”) in a suitable solvent to form the inclusion complex of R-(+)-lansoprazole with R-(+)-BINOL;
(b) removing the inclusion complex of R-(+)-lansoprazole with R-(+)-BINOL prepared in step (a) from the solvent and adding a mixture of organic solvents;
(c) crystallizing the R-(+)-lansoprazole-R-(+)-BINOL inclusion complex from the mixture of organic solvents;
(d) enriching the R-(+)-lansoprazole-R-(+)-BINOL inclusion complex to obtain a purity of more than about 97% e.e;
(f) cleaving the enriched R-(+)-lansoprazole-R-(+)BINOL inclusion complex;
(g) separating the R-BINOL and R-(+)-lansoprazole; and
(h) isolating the R-(+)-lansoprazole.

4. The process of claim 3, wherein the mixture of organic solvents is a mixture of an aromatic and an aliphatic solvent.

5. The process of claim 4, wherein the mixture of solvents is toluene and hexane.

6. The process of claim 4 wherein the ratio of aromatic to aliphatic solvent is about 1:1 to about 5:0.5

7. The process of claim 6 wherein the ratio of aromatic to aliphatic solvent is about 2:1 to about 5:1.

8. The process of claim 6 wherein the ratio of aromatic to aliphatic solvent is about 4:1.

9. The process of claim 3, wherein the enriched (R)-lansoprazole-R-(+)BINOL inclusion complex is cleaved with an aqueous ammonia solution to give R-(+)-lansoprazole.

10. The process of claim 3, wherein R-(+)-lansoprazole is adsorbed on pharmaceutically acceptable polymer.

11. The process of claim 10 wherein the pharmaceutically acceptable polymer is a water soluble polymer.

12. The process of claim 11 wherein the pharmaceutically acceptable polymer is polyvinylpyrrolidone.

13. A pharmaceutical dosage form comprising the R-(+)-lansoprazole prepared according to claim 3 and at least one additional pharmaceutically excipient.
